(19) United States
(12) Patent Application Publication
(10) Pub. No.: US 2015/0080795 A1
(43) Pub. Date: Mar. 19, 2015

(54) DEVICES, SYSTEMS AND METHODS FOR PERFORMING ATHERECTOMY AND SUBSEQUENT BALLOON ANGIOPLASTY WITHOUT EXCHANGING DEVICES

(71) Applicant: Cardiovascular Systems, Inc., St. Paul, MN (US)

(72) Inventors: Richard C. Mattison, Zimmerman, MN (US); Robert E. Kohler, Lake Elmo, MN (US); Victor L. Schoenle, Greenfield, MN (US)

(21) Appl. No.: 14/339,933

(22) Filed: Jul. 24, 2014

Related U.S. Application Data
(60) Provisional application No. 61/858,881, filed on Jul. 26, 2013.
(60) Provisional application No. 61/861,041, filed on Aug. 1, 2013.

(51) Int. Cl. A61B 17/3207; A61M 25/10

(52) U.S. Cl. CPC A61B 17/320758; A61M 25/10184; A61M 25/104; A61B 2017/320004
USPC 604/99.01; 606/180; 604/96.01; 604/103.01; 604/103.05

(57) ABSTRACT

The present invention is directed in various methods, devices and systems relating to providing a balloon on a sheath in combination with orbital atherectomy in order reduce the number of steps in the procedure. In certain embodiments, the balloon comprises adjunctive low pressure balloon for prevention of vessel trauma during dilatation.

### CROSS-REFERENCE TO RELATED APPLICATIONS

This application claims priority to App. Ser. No. 61/858,881, entitled “Devices, Systems and Methods for an Atherectomy Device with Balloon Tipped Saline Sheath,” filed Jul. 26, 2013, and to App. Ser. No. 61/861,041, entitled “Devices, Systems and Methods for an Atherectomy Device with Balloon Tipped Saline Sheath and Having Adjunctive Low Pressure Balloon,” filed Aug. 1, 2013, the entire contents of each of which are hereby incorporated by reference.

### FIELD OF THE INVENTION

The present disclosure generally relates to methods, devices and systems relating to removing occlusions from vessels. More specifically, the present invention comprises providing a balloon

### DESCRIPTION OF THE RELATED ART

A variety of techniques and instruments have been developed for use in the removal or repair of tissue in arteries and similar body passageways, e.g., biological conduits. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

### BRIEF SUMMARY OF THE INVENTION

The present invention is directed in various methods, devices and systems relating to providing a balloon on a sheath in combination with orbital atherectomy in order reduce the number of steps in the procedure. In certain embodiments, the balloon comprises adjunctive low pressure balloon for prevention of vessel trauma during dilatation.

### BRIEF DESCRIPTION OF THE DRAWINGS

FIG. 1 illustrates a perspective view of one embodiment of the present invention;

FIG. 2 illustrates a front view of one embodiment of the present invention;

FIG. 3 illustrates a side view of one embodiment of the present invention;

FIG. 4 illustrates a cutaway side view of one embodiment of the present invention;

FIG. 5 illustrates a side view of one embodiment of the present invention; and

FIG. 6 illustrates a front view of one embodiment of the present invention.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

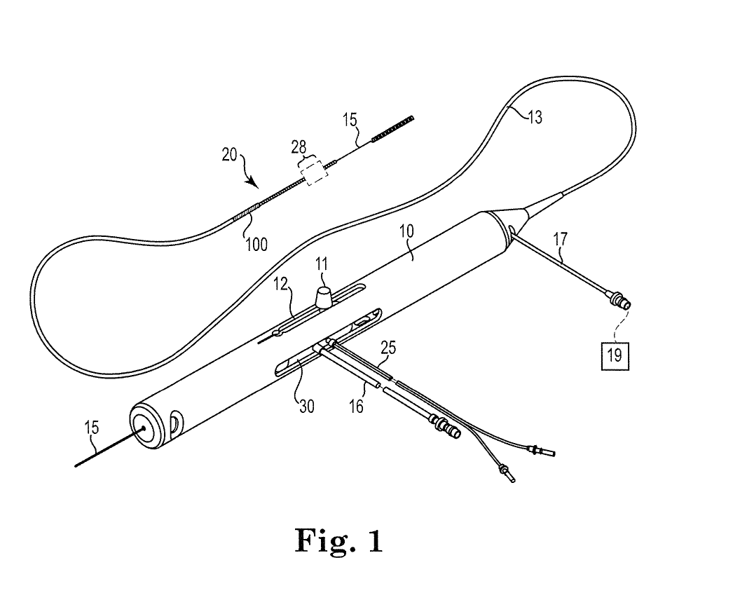

FIG. 1 illustrates an exemplary rotational atherectomy device of the invention. The device includes a handle portion 10, an elongated, flexible drive shaft 20 having an abrasive section 28, as will be readily understood by the skilled artisan, and an elongated catheter 13 extending distally from the handle portion 10. The abrasive section 28 is disposed on the drive shaft 20. The catheter 13 has a lumen in which the length of the drive shaft 20 may be disposed, including, in certain embodiments, the abrasive section 28 and, when present, a short section distal to the abrasive section 28. The drive shaft 20 also contains an inner lumen, permitting the drive shaft 20 to be advanced and rotated over a guide wire 15. A fluid supply line 17 may be provided for introducing a cooling and lubricating solution (typically saline or another biocompatible fluid) into the catheter 13 from a cooling and lubrication solution reservoir (not shown but as well understood by the skilled artisan). A separate inflation media reservoir 19 may be included for providing inflation media, preferably saline, to an inflatable balloon 100 on the tip of the catheter 13.

The handle 10 desirably contains an electric motor (or similar rotational drive mechanism, e.g., a turbine) for rotating the drive shaft 20 at low or high speeds. The handle 10 typically may be connected to a power source, such as compressed air delivered through a tube 16. A pair of fiber optic cables 25 may also be provided for monitoring the speed of rotation of the turbine and drive shaft 20. Details regarding such handles and associated instrumentation are well known in the industry. The handle 10 also desirably includes a control knob 11 for advancing and retracting the electric motor, or equivalent, and the drive shaft 20 with respect to the catheter 13 and the body of the handle.

The inflatable balloon 100 mounted on the tip of catheter 13 allows for rotational atherectomy to proceed with abrasive section 28, followed by advancement of the catheter 13 such that the inflatable balloon is within the treatment region of the vessel. Balloon 100 is illustrated in the various Figures as attached to outer surface of catheter 13 and as completely surrounding the outer surface of catheter 13 and this is the preferred embodiment. However, the skilled artisan will recognize that alternate embodiments may be provided to achieve the requisite dilatation. Inflation of the balloon 100 by introduction of the inflation media from inflation reservoir 19 is accomplished as is well understood by the skilled artisan to achieve dilatation of the treatment region post-atherectomy procedure. Such an arrangement eliminates the need to withdraw the atherectomy device and, subsequently, advance a balloon device back to the treatment site for dilatation. Thus, embodiments of the present invention eliminates the need for the extra time to remove the atherectomy device and insert and position a balloon. In addition, radiopaque dye and subsequent radiation exposure will be reduced.

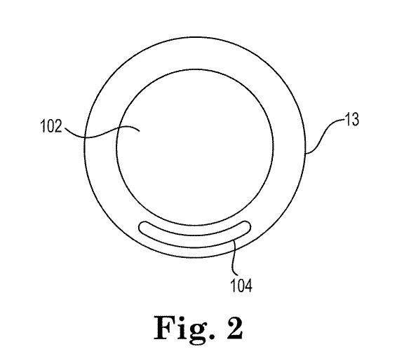

In the embodiment of FIG. 1, catheter 13 may comprise two lumens such as illustrated in FIG. 2. Catheter 13 of FIG. 2 comprises a drive shaft lumen 102 wherein the drive shaft 20 is rotatable and translatable. Catheter 13 also comprises an inflation medium lumen 104. Inflation medium lumen 104 is in fluid communication with inflation medium reservoir 19 and inflatable balloon 100, serving as the conduit for inflation medium, e.g., saline, to be controllably moved through lumen 104 from the reservoir 19 to the inflatable balloon 100 by means that are well known to the skilled artisan.

A system according to FIGS. 1 and 2 may comprise:
a rotatable drive shaft having an abrasive section and means for rotating and translating the drive shaft;
a catheter collinear with the rotatable drive shaft and comprising
   a distal end;
   an inflatable balloon mounted proximate the distal end of the catheter;
   a drive shaft lumen wherein the drive shaft is rotatable and translatable; and
   an inflation lumen separated from the drive shaft lumen; and
an inflation media reservoir comprising an inflation medium, wherein the inflation lumen is in fluid communication with the inflation lumen and the inflatable balloon, and wherein a portion of the inflatable balloon may be pulled proximally within the drive shaft lumen to adjust the length of the inflated balloon exposed outside of the drive shaft lumen.

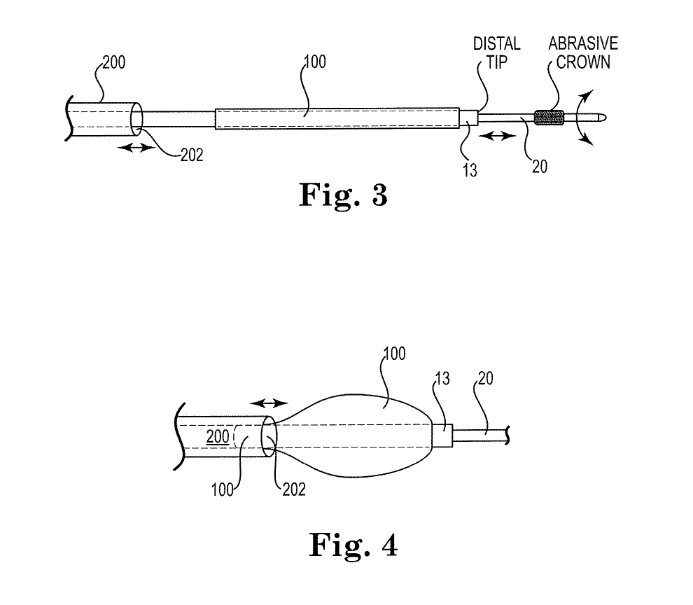

FIG. 3 provides an alternative embodiment wherein the device of FIGS. 1 and 2 further comprises sheath 200 having a lumen therethrough 202. Catheter 13, with balloon 100 disposed on or near catheter's distal tip, is rotatably and translatably disposed within lumen 202. Catheter 13 further comprises drive shaft lumen 102 and inflation media lumen 104, which is in fluid communication with inflation media reservoir 19 and inflation balloon 100 as shown and discussed in connection with FIGS. 1 and 2. Drive shaft 20 is, as also discussed above, rotatably and translatably disposed within drive shaft lumen 102.

In the embodiment of FIG. 3, sheath 200 with both drive shaft 20 and catheter 13 drawn proximally into lumen 202, both being collinear, may be advanced to a point proximal of the treatment region, e.g., occlusion. Distal advancement of the drive shaft 20 allows distal translation of the drive shaft 20 and the abrasive section 28 (shown in FIG. 3 as an exemplary eccentric abrasive crown) out of lumen 202 and, in certain embodiments, out of the drive shaft lumen 102 of catheter 13. This allows the operator to initiate and complete the rotational atherectomy procedure using the exemplary eccentric abrasive crown. Once complete, the operator may simply advance catheter 13 so that the inflatable balloon 100 is positioned within the treatment region and initiate dilatation of same. The drive shaft 20 and exemplary abrasive crown may be left in place during dilatation or, alternatively, may be proximally translated into the drive shaft lumen 102. Once dilatation is complete, the balloon 100 is deflated and catheter 13 and drive shaft 20 are once again moved proximally into the lumen 202 of sheath 200 for removal. A further use of lumen 202 may comprise dye or drug injection into the lumen at the localized treatment region, thereby eliminating the need for long introducers or settling for diffuse dye coverage or drug uptake.

FIG. 4 provides a variation of the collinear device illustrated in FIG. 3, wherein sheath 200 may be used to adjust the inflated length of the inflatable balloon 100. Here, as shown, catheter 13 is drawn proximally into lumen 202 so that at least a portion of the inflated balloon 100 is pulled within lumen 202, thereby shortening the overall length of exposure of inflated balloon 100 to the vessel wall. Alternatively, sheath 200 may be advanced distally to enable movement of at least a portion of the balloon to enter lumen 202. The length that the inflated balloon is shortened by the process of FIG. 4 is equal to the length of the balloon that is pulled within the lumen 202. As will be appreciated, this shortening of the balloon 100 may be initiated before inflation of balloon 100, during inflation of balloon 100 and/or after inflation of balloon 100. Lumen 202 may also be used to deploy self-expanding stents, stent graphs or distal filters.

Thus, a system according to the embodiment of FIG. 4 may comprise:

A system for atherectomy and subsequent dilatation, comprising:
a rotatable drive shaft having an abrasive section and means for rotating and translating the drive shaft;
a catheter collinear with the rotatable drive shaft and comprising
   a distal end;
   an inflatable balloon mounted proximate the distal end of the catheter;
   a drive shaft lumen wherein the drive shaft is rotatable and translatable; and
   an inflation lumen separated from the drive shaft lumen;
an inflation media reservoir comprising an inflation medium, wherein the inflation lumen is in fluid communication with the inflation lumen and the inflatable balloon; and

a sheath having a lumen therethrough, wherein the catheter and drive shaft are collinear with the sheath and wherein the catheter is rotatable and translatable within the sheath, wherein a portion of the inflatable balloon may be pulled proximally within the lumen of the sheath to adjust the length of the inflated balloon exposed outside of the lumen of the sheath.

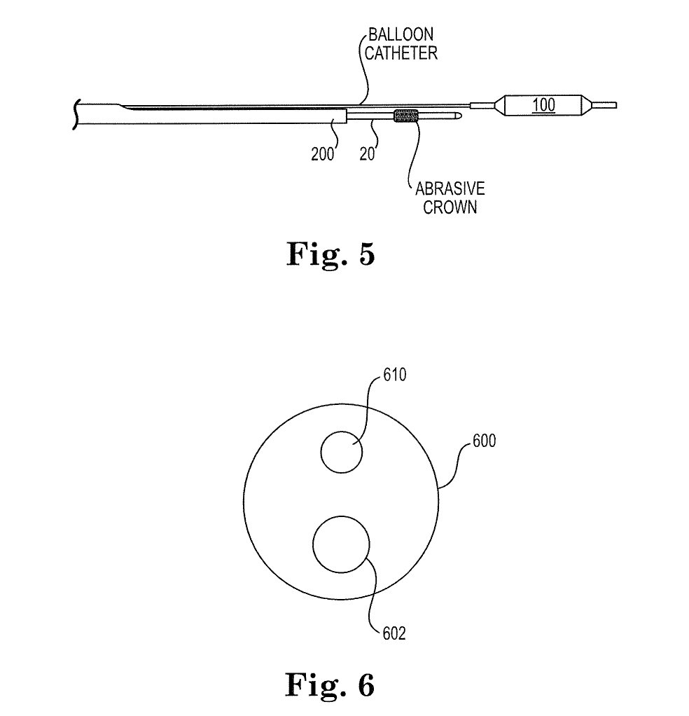

FIG. 5 provides another embodiment wherein the inflatable balloon 100 and drive shaft 20 are not collinear. Here, sheath 200 comprises at least two separate lumens as illustrated in FIG. 6: a drive shaft lumen 602 wherein the drive shaft and abrasive section 28 (illustrated as exemplary eccentric abrasive crown) are rotatably and translatably disposed; and an inflatable balloon lumen 610 wherein the balloon catheter and inflatable balloon affixed to the distal end of the balloon catheter, are rotatably and translatably disposed.

In this embodiment, sheath 200 is positioned proximal to the treatment region, e.g., the occlusion, with balloon catheter and balloon 100 and drive shaft 20 and abrasive crown disposed within lumens 610 and 602, respectively. The operator may extend the drive shaft 20 distally and out of the drive shaft lumen 602 to accomplish the rotational atherectomy procedure with the exemplary abrasive crown. When completed, the drive shaft 20 and exemplary abrasive crown may be proximally pulled back into drive shaft lumen 602. At this point, the operator may extend the balloon catheter and balloon 100 distally and out of the balloon lumen 610 to the treatment region to inflate balloon 100 as described above and accomplish the required dilatation. When dilatation is complete, the operator deflates the balloon and withdraws the now-deflated balloon proximally into the balloon lumen 610 for removal.

FIG. 5 illustrates the distal portion of the sheath 200 as comprising a partial cutaway section. Other embodiments of sheath 200 do not include this partially cutaway section and comprise sheath 200 as illustrated in FIGS. 3 and 4.

In any of the above embodiments described above, or the equivalent, the balloon 100 may be micro-porous to enable delivery of a therapeutic agent to the vessel wall, e.g., an anti-restinosis agent. The balloon may also be coated with a drug for delivery of the therapeutic agent to the vessel wall. The balloon 100 of the present invention, when used for low-pressure dilatation comprises an acceptable fluid loss during the inflation cycle. Stent deployment may be an option in all embodiments described herein relating to balloon inflation.

As discussed, generally, atherectomy procedures are followed up with a balloon procedure to remodel the artery and to provide a larger lumen internal diameter (ID). Rotational atherectomy allows for subsequent low-pressure balloon dilatations. Low-pressure balloon dilatations of e.g., 2 to 4 atmospheres of pressure are much less traumatic to the vessel wall than the more typical 10 to 13 atmospheres of pressure. A preferred low-pressure balloon range using the devices and methods of the present invention comprises 1 to 8 atmospheres, a more preferred range comprises 2 to 6 atmospheres, and a still more preferred range comprises 2 to 4 atmospheres of pressure.

Various embodiments of the present invention may be incorporated into a rotational atherectomy system as described generally in U.S. Pat. No. 6,494,890, entitled “ECCENTRIC ROTATIONAL ATHERECTOMY DEVICE,” which is incorporated herein by reference. Additionally, the disclosure of the following co-owned patents or patent applications are herein incorporated by reference in their entireties: U.S. Pat. No. 6,295,712, entitled “ROTATIONAL ATHERECTOMY DEVICE”; U.S. Pat. No. 6,132,444, entitled “ECCENTRIC DRIVE SHAFT FOR ATHERECTOMY DEVICE AND METHOD FOR MANUFACTURE”; U.S. Pat. No. 6,638,288, entitled “ECCENTRIC DRIVE SHAFT FOR ATHERECTOMY DEVICE AND METHOD FOR MANUFACTURE”; U.S. Pat. No. 5,314,438, entitled “ABRASIVE DRIVE SHAFT DEVICE FOR ROTATIONAL ATHERECTOMY”; U.S. Pat. No. 6,217,595, entitled “ROTATIONAL ATHERECTOMY DEVICE”; U.S. Pat. No. 5,554,163, entitled “ATHERECTOMY DEVICE”; U.S. Pat. No. 7,507,245, entitled “ROTATIONAL ANGIOPLASTY DEVICE WITH ABRASIVE CROWN”; U.S. Pat. No. 6,129,734, entitled “ROTATIONAL ATHERECTOMY DEVICE WITH RADIALLY EXPANDABLE PRIME MOVER COUPLING”; U.S. Pat. No. 8,597,313, entitled “ECCENTRIC ABRADING HEAD FOR HIGH-SPEED ROTATIONAL ATHERECTOMY DEVICES”; U.S. Pat. No. 8,439,937, entitled “SYSTEM, APPARATUS AND METHOD FOR OPENING AN OCCLUDED LESION”; U.S. Pat. Pub. No. 2009/0299392, entitled “ECCENTRIC ABRADING ELEMENT FOR HIGH-SPEED ROTATIONAL ATHERECTOMY DEVICES”; U.S. Pat. Pub. No. 2010/0198239, entitled “MULTI-MATERIAL ABRADING HEAD FOR ATHERECTOMY DEVICES HAVING LATERALLY DISPLACED CENTER OF MASS”; U.S. Pat. Pub. No. 2010/0036402, entitled “ROTATIONAL ATHERECTOMY DEVICE WITH PRE-CURVED DRIVE SHAFT”; U.S. Pat. Pub. No. 2009/0299391, entitled “ECCENTRIC ABRADING AND CUTTING HEAD FOR HIGH-SPEED ROTATIONAL ATHERECTOMY DEVICES”; U.S. Pat. Pub. No. 2010/0100110, entitled “ECCENTRIC ABRADING AND CUTTING HEAD FOR HIGH-SPEED ROTATIONAL ATHERECTOMY DEVICES”; U.S. Design Pat. No. D610258, entitled “ROTATIONAL ATHERECTOMY ABRASIVE CROWN”; U.S. Design Pat. No. D6107102, entitled “ROTATIONAL ATHERECTOMY ABRASIVE CROWN”; U.S. Pat. Pub. No. 2009/0306689, entitled “BIDIRECTIONAL EXPANDABLE HEAD FOR ROTATIONAL ATHERECTOMY DEVICE”; U.S. Pat. Pub. No. 2010/0211088, entitled “ROTATIONAL ATHERECTOMY SEGMENTED ABRADING HEAD AND METHOD TO IMPROVE ABRADING EFFICIENCY”; U.S. Pat. Pub. No. 2013/0018398, entitled “ROTATIONAL ATHERECTOMY DEVICE WITH ELECTRIC MOTOR”; and U.S. Pat. No. 7,666,202, entitled “ORBITAL ATHERECTOMY DEVICE GUIDE WIRE DESIGN.” It is contemplated by this invention that the features of one or more of the embodiments of the present invention may be combined with one or more features of the embodiments of atherectomy devices described therein.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A system for atherectomy and subsequent dilatation, comprising:
a rotatable drive shaft having an abrasive section and means for rotating and translating the drive shaft;
a catheter collinear with the rotatable drive shaft and comprising
a distal end;
an inflatable balloon mounted proximate the distal end of the catheter;
a drive shaft lumen wherein the drive shaft is rotatable and translatable; and
an inflation lumen separated from the drive shaft lumen; and
an inflation media reservoir comprising an inflation medium, wherein the inflation lumen is in fluid communication with the inflation lumen and the inflatable balloon.

2. The system of claim 1, further comprising a sheath having a lumen therethrough, wherein the catheter and drive shaft are collinear with the sheath and wherein the catheter is rotatable and translatable within the sheath.

3. The system of claim 2, wherein a portion of the inflatable balloon may be pulled proximally within the lumen of the sheath to adjust the length of the inflated balloon exposed outside of the lumen of the sheath.

4. The system of claim 3, wherein the inflatable balloon is inflated after a portion of the inflatable balloon is pulled proximally within the lumen of the sheath.

5. The system of claim 3, wherein the inflatable balloon is inflated and subsequently a portion of the inflated balloon is pulled proximally within the lumen of the sheath.

6. The system of claim 1, wherein the abrasive element comprises an eccentric abrasive crown.

7. The system of claim 1, wherein the inflatable balloon is inflated to an adjunctive low pressure within the range of 1 to 8 atmospheres of pressure.

8. The system of claim 1, wherein the inflatable balloon is inflated to an adjunctive low pressure within the range of 2 to 6 atmospheres of pressure.

9. The system of claim 1, wherein the inflatable balloon is inflated to an adjunctive low pressure within the range of 2 to 4 atmospheres of pressure.

10. The system of claim 2, wherein the inflatable balloon is inflated to an adjunctive low pressure within the range of 1 to 8 atmospheres of pressure.

11. The system of claim 2, wherein the inflatable balloon is inflated to an adjunctive low pressure within the range of 2 to 6 atmospheres of pressure.

12. The system of claim 2, wherein the inflatable balloon is inflated to an adjunctive low pressure within the range of 2 to 4 atmospheres of pressure.

13. The system of claim 1, wherein the inflatable balloon is microporous.

14. The system of claim 1, wherein the inflatable balloon is capable of delivering a therapeutic agent to the vessel wall.

15. The system of claim 13, wherein the inflatable balloon is capable of delivering a therapeutic agent to the vessel wall.

16. A system for atherectomy and subsequent dilatation, comprising:
a rotatable drive shaft having an abrasive section and means for rotating and translating the drive shaft;
a catheter collinear with the rotatable drive shaft and comprising
a distal end;
an inflatable balloon mounted proximate the distal end of the catheter;
a drive shaft lumen wherein the drive shaft is rotatable and translatable; and
an inflation lumen separated from the drive shaft lumen; and
an inflation media reservoir comprising an inflation medium, wherein the inflation lumen is in fluid communication with the inflation lumen and the inflatable balloon, and wherein a portion of the inflatable balloon may be pulled proximally within the drive shaft lumen to adjust the length of the inflated balloon exposed outside of the drive shaft lumen.

17. The system of claim 16, wherein the inflatable balloon is inflated after a portion of the inflatable balloon is pulled proximally within the drive shaft lumen.

18. The system of claim 16, wherein the inflatable balloon is inflated and subsequently a portion of the inflated balloon is pulled proximally within the drive shaft lumen.

19. The system of claim 16, wherein the inflatable balloon is inflated to an adjunctive low pressure within the range of 1 to 8 atmospheres of pressure.

20. The system of claim 16, wherein the inflatable balloon is inflated to an adjunctive low pressure within the range of 2 to 6 atmospheres of pressure.

21. The system of claim 16, wherein the inflatable balloon is inflated to an adjunctive low pressure within the range of 2 to 4 atmospheres of pressure.

22. The system of claim 16, wherein the abrasive element comprises an eccentric abrasive crown.

23. The system of claim 16, wherein the inflatable balloon is microporous.

24. The system of claim 16, wherein the inflatable balloon is capable of delivering a therapeutic agent to the vessel wall.

25. The system of claim 23, wherein the inflatable balloon is capable of delivering a therapeutic agent to the vessel wall.

26. A system for atherectomy and subsequent dilatation, comprising:
a rotatable drive shaft having an abrasive section and means for rotating and translating the drive shaft;
an inflatable balloon catheter having a lumen therethrough and in fluid communication with an inflation media reservoir;
an inflatable balloon mounted proximate the distal end of the inflatable balloon catheter, wherein the inflatable balloon catheter and the rotatable drive shaft are not collinear and wherein the inflatable balloon is in fluid communication with the lumen of the inflatable balloon catheter; and
a sheath comprising:
a drive shaft lumen wherein the drive shaft is rotatable and translatable; and
an inflatable balloon lumen separated from the drive shaft lumen, wherein the inflatable balloon catheter is rotatable and translatable.

27. The system of claim 26, wherein the inflatable balloon is inflated to an adjunctive low pressure within the range of 1 to 8 atmospheres of pressure.

28. The system of claim 26, wherein the inflatable balloon is inflated to an adjunctive low pressure within the range of 2 to 6 atmospheres of pressure.

29. The system of claim 26, wherein the inflatable balloon is inflated to an adjunctive low pressure within the range of 2 to 4 atmospheres of pressure.

30. The system of claim 26, wherein the inflatable balloon is microporous.

31. The system of claim 26, wherein the inflatable balloon is capable of delivering a therapeutic agent to the vessel wall.

32. The system of claim 30, wherein the inflatable balloon is capable of delivering a therapeutic agent to the vessel wall.

33. A method for performing atherectomy and subsequent dilatation without exchanging the atherectomy device for the inflatable balloon device, comprising:
providing a drive shaft with an abrasive section;
providing a catheter having an inflatable balloon disposed proximate the distal end of the catheter and a lumen therethrough wherein the drive shaft is rotatable and translatable;
extending the drive shaft distally out of the catheter lumen to the treatment region and rotating the drive shaft and abrasive section within the treatment region;
extending the catheter proximally to position the inflatable balloon within the treatment region;
inflating the inflatable balloon to achieve at least one dilatation within the treatment region;
deflating the inflatable balloon and pulling the inflatable balloon and drive shaft proximally into the catheter lumen for removal.
